(19) United States
(12) Patent Application Publication
(10) Pub. No.: US 2025/0127743 A1
(43) Pub. Date: Apr. 24, 2025

(54) USE OF BRANCHED CHAIN FATTY ACIDS (BCFAS) FOR THE TREATMENT OF INTESTINAL INFLAMMATION

(71) Applicants: INSTITUT NATIONAL DE AL SANTÉ ET DE LA RECHERCHE MÉDICALE, Paris (FR); CENTRE HOSPITALIER UNIVERSITAIRE DE ROUEN, Rouen (FR); UNIVERSITÉ DE ROUEN NORMANDIE, Mont-Saint-Aignan (FR)

(72) Inventors: David RIBET, Rouen (FR); Pierre DECHELOTTE, Rouen (FR); Chaima EZZINE, Rouen (FR)

(21) Appl. No.: 18/687,496

(22) PCT Filed: Aug. 30, 2022
(86) PCT No.: PCT/EP2022/074122

§ 371 Date: Feb. 28, 2024

(30) Foreign Application Priority Data
Aug. 31, 2021 (EP) 21306182.3

(51) Int. Cl. A61K 31/20; A61K 31/19; A61K 45/06; A61P 1/14; A61P 29/00

(52) U.S. Cl. CPC A61K 31/20; A61K 31/19; A61K 45/06; A61P 1/14; A61P 29/00

(57) ABSTRACT

The gut microbiota produces a wide variety of metabolites, which interact with intestinal cells by modulating either gene transcription or post-translational modifications of gut proteins. The effect of gut commensal bacteria on SUMOylation, an essential ubiquitin-like modification in intestinal physiology, remains however unknown. Here, the inventors show that branched chain fatty acids (BCFAs) increase protein SUMOylation in different intestinal cell lines. They demonstrated that the hyperSUMOylation induced by BCFAs inhibits the activation of the NF-κB pathway by blocking the degradation of the inhibitory factor IκBα in response to TNFα. This results in a decrease in pro-inflammatory cytokines expression as well as a decrease in intestinal epithelial permeability in response to TNFα. Accordingly, the present invention relates to the use of Branched Chain Fatty Acids (BCFAs) for the treatment of diseases associated with intestinal inflammation such as Inflammatory Bowel Diseases and Irritable Bowel Syndrome.

### FIELD OF THE INVENTION

The present invention is in the field of medicine, in particular gastroenterology.

### BACKGROUND OF THE INVENTION

The gut microbiota produces a wide variety of metabolites diffusing to the intestinal mucosa and modulating intestinal cell activities (Gasaly et al., 2021). Some of these metabolites may even cross the intestinal barrier and reach distant organs via the bloodstream or via nerve communications. Fatty acids constitute a major class of metabolites produced by intestinal bacteria. They include the so-called Short Chain Fatty Acids (SCFAs), which are carboxylic acids with aliphatic tails of 1 to 6 carbons (Koh et al., 2016). Acetic, butyric and propionic acids are the main SCFAs produced in the human colon and derive mostly from the anaerobic catabolism of dietary fibers by intestinal bacteria (Parada Venegas et al., 2019; Blaak et al., 2020). Branched Chain Fatty Acids (BCFAs), such as isobutyric, isovaleric or 2-methylbutyric acids, constitute another class of bacteria-derived fatty acids with one or more methyl branches on the carbon chain. BCFA mostly derive from the breakdown of proteins by intestinal bacteria, and more particularly from the catabolism of branched-chain amino-acids (valine, leucine and isoleucine, producing isobutyrate, isovalerate or 2-methylbutyrate, respectively) (Blachier et al., 2007).

Fatty acids regulate intestinal cell activities by various mechanisms. They may bind to specific receptors expressed on intestinal cells, such as GPR41/FFAR3, GPR43/FFAR2 and GPR109A, and activate various signaling pathways (Kimura et al., 2020). Fatty acids may also directly enter into intestinal cells by passive diffusion or by facilitated transport. Fatty acids are weak organic acids, which exist in solution either as acidic or basic forms. Only the acidic (uncharged) forms may passively diffuse across the plasma membrane, whereas the basic (negatively charged) forms are uptaken via specific transporters such as MCT1, MCT4, SMCT1 or SMCT2 (Sivaprakasam et al., 2017). Once in intestinal cells, they participate to the cell metabolism. For example, colonocytes were shown to use butyrate as a major energy source or, alternatively, isobutyrate when butyrate availability is low (Roediger, 1980; Jaskiewicz et al., 1996). Finally, fatty acids may regulate intestinal cells activity by interfering with post-translational modification such as neddylation (Kumar et al., 2007; Kular et al., 2009). The impact of fatty acids on other ubiquitin-like modifications in intestinal cells has not been described yet.

SUMOylation is an ubiquitin-like modifications consisting in the covalent addition of SUMO (Small Ubiquitin-like MOdifier) peptides to target proteins. Five SUMO paralogs have been identified in humans that share 45-97% sequence identity. SUMO1, SUMO2 and SUMO3, which are the most studied paralogs, can be conjugated to both overlapping and distinct sets of proteins (Flotho and Melchior, 2013). The conjugation of SUMO to lysine residues of target proteins is catalysed by an enzymatic machinery composed of one E1 enzyme (SAE1/SAE2), one E2 enzyme (UBC9) and several E3 enzymes (Cappadocia and Lima, 2018). SUMOylation is a reversible modification as the isopeptide bond between SUMO and its target can be cleaved by specific proteases called deSUMOylases (Kunz et al., 2018). The consequences of SUMO conjugation on target proteins are very diverse and include changes in protein localization, stability, activity or interactions with other cellular components (Flotho and Melchior, 2013; Zhao, 2018; Chang and Yeh, 2020).

SUMOylation plays essential roles in intestinal physiology as it limits detrimental inflammation while participating to tissue integrity maintenance (Demarque et al., 2011; Karhausen et al., 2021). Interestingly, several intestinal bacterial pathogens were shown to interfere with epithelial cell SUMOylation (Ribet and Cossart, 2018). Listeria monocytogenes, for example, secretes a pore-forming toxin triggering the degradation of the host cell E2 SUMO enzyme and the rapid loss of SUMO-conjugated proteins (Ribet et al., 2010; Impens et al., 2014). Salmonella enterica serovar Typhimurium also targets the host E2 SUMO enzymes during infection by inhibiting its translation via miRNA-based mechanisms (Verma et al., 2015). Shigella flexneri, finally, similarly switches off the SUMOylation machinery by triggering a calpain-dependent cleavage of the SUMO E1 enzyme SAE2 subunit in infected cells (Lapaquette et al., 2017). In contrast to these examples of pathogens dampening intestinal cell SUMOylation, the impact of gut commensal bacteria on the SUMOylation of intestinal proteins remains unknown.

### SUMMARY OF THE INVENTION

The present invention is defined by the claims. In particular, the present invention relates to the use of Branched Chain Fatty Acids (BCFAs) for the treatment of pathologies characterized by intestinal inflammation such as inflammatory bowel diseases and irritable bowel syndrome.

### DETAILED DESCRIPTION OF THE INVENTION

The gut microbiota produces a wide variety of metabolites, which interact with intestinal cells and participate to host physiology. These metabolites regulate intestinal cell activities by modulating either gene transcription or post-translational modifications of gut proteins. The effect of gut commensal bacteria on SUMOylation, an essential ubiquitin-like modification in intestinal physiology, remains however unknown. Here, the inventors show that short chain fatty acids (SCFAs) and branched chain fatty acids (BCFAs) increase protein SUMOylation in different intestinal cell lines. They demonstrate that the hyperSUMOylation induced by SCFAs/BCFAs is pH-dependent and results from the passive diffusion of these fatty acids through intestinal cell plasma membranes. Once inside cells, SCFAs/BCFAs trigger the inactivation of deSUMOylases, which are enzymes involved in the deconjugation of SUMO, via the induction of an oxidative stress. This inactivation favors SUMO-conjugation reactions and promote the hyperSUMOylation of chromatin-bound proteins. In order to determine the impact of these modifications on intestinal physiology, the inventors focused on the NF-κB signaling pathway, a key player in inflammation known to be regulated by SUMOylation. They demonstrated that the hyperSUMOylation induced by SCFAs/BCFAs inhibits the activation of the NF-κB pathway by blocking the degradation of the inhibitory factor IκBα in response to TNFα. This results in a decrease in pro-inflammatory cytokines expression such as IL8 or CCL20, as well as a decrease in intestinal epithelial permeability in response to TNFα. Together, the inventors reveal that fatty acids produced by gut commensal bacteria regulate intestinal physiology by modulating SUMOylation and illustrate a new mechanism of dampening of host inflammatory responses by the gut microbiota.

Accordingly, the first object of the present invention relates to a method of treating an intestinal inflammation in a patient in need thereof comprising administering to the patient a therapeutically effective amount of branched chain fatty acids.

As used herein the term “intestinal inflammation” has its general meaning in the art and refers to a chronic disease that causes inflammation in the small intestine or large intestine.

In some embodiment, the present invention relates to a method of treating an inflammatory bowel disease in a patient in need thereof comprising administering to the patient a therapeutically effective amount of branched chain fatty acids.

As used herein the term “inflammatory bowel disease” has its general meaning in the art and refers to any inflammatory disease that affects the bowel. The term includes but is not limited to ulcerative colitis, Crohn's disease, especially Crohn's disease in a state that affect specifically the colon with or without ileitis, microscopic colitis (lymphocytic colitis and collagenous colitis), infectious colitis caused by bacteria or by virus, radiation colitis, ischemic colitis, pediatric colitis, undetermined colitis, functional bowel disorders (and by extension functional gastrointestinal disorders) and other states of digestive microinflammation, including irritable bowel syndrome (described symptoms without evident anatomical abnormalities).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In particular the method of the present invention is particularly suitable for increasing protein SUMOylation in intestinal cells, for decreasing expression of pro-inflammatory cytokines expression (e.g. IL8 or CCL20), and for decreasing intestinal epithelial permeability triggered by inflammation (e.g. TNFα).

As used herein, the term “branched chain fatty acid” or “BCFA” has its general meaning in the art and refers to a fatty acid containing a carbon constituent branched on the main carbon chain of the fatty acid. Typically, the BCFAs of the present invention are selected from the group consisting of iso- and anteiso-methyl-branched fatty acids. Iso-methyl branched fatty acids have the branch point on the penultimate carbon (one from the end or (ω-1)), while anteiso-methyl-branched fatty acids have the branch point on the ante-penultimate carbon atom (two from the end or (ω-2)). More particularly, the BCFA of the present invention can be a branched form of a fatty acid with a short acyl chain that typically comprises 3 to 7 carbons in the acyl chain. These BCFAs may be saturated or unsaturated or mixtures thereof. Thus, in some embodiments, the BCFA is a branched form of a fatty acid selected from fatty acids comprising 7 or less carbon atoms, 6 or less carbon atoms, 5 or less carbon atoms, 4 or less carbon atoms. In some embodiments, the BCFA is selected from the group consisting of isobutyric, isovaleric, 2-methyl-butyric acids and mixture thereof. More particularly, the BCFA are used in their acidic form.

According to the present invention, the method of the present invention comprises locally administering the BCFA to the rectum, colon and/or terminal ileum of the patient. In some embodiments, the BCFA are administered orally, by means of a unit dosage form that selectively releases BCFA in the terminal ileum and/or colon of the patient. In some embodiments, the BCFA are effectively administered to the colon by rectal administration of an enema formulation or rectal foam comprising BCFA. In some embodiments, the BCFA are delivered to the ileum or colon of the patient by administration of an enterically coated unit dosage form. Pharmaceutical preparations suitable for oral administration may, for example, be in the form of tablets, capsules, syrups, solutions and drinkable suspensions, drops, granulates, preparations for sublingual administration or gastrointestinal formulations, or preparations administrable parenterally, nutritional composition, dietary supplements, functional foods, and nutraceuticals. In some embodiments, the BCFA are administered to the subject in the form of a nutritional composition. As used herein, the term “nutritional composition” means a composition which nourishes a subject. This nutritional composition usually includes a lipid or fat source and optionally a protein source and/or optionally a carbohydrate source and/or optionally minerals and vitamins. Preferably, the nutritional composition is for oral use and thus represents a food composition. In some embodiments, the food composition is selected from complete food compositions, food supplements, nutraceutical compositions, and the like. The composition of the present invention may be used as a food ingredient and/or feed ingredient. The food ingredient may be in the form of a solution or as a solid—depending on the use and/or the mode of application and/or the mode of administration. As used herein, the term “food” refers to liquid (i.e. drink), solid or semi-solid dietetic compositions, especially total food compositions (food-replacement), which do not require additional nutrient intake or food supplement compositions. Food supplement compositions do not completely replace nutrient intake by other means. As used herein the term “food ingredient” or “feed ingredient” includes a formulation which is or can be added to functional foods or foodstuffs as a nutritional supplement. By “nutritional food” or “nutraceutical” or “functional” food, is meant a foodstuff which contains ingredients having beneficial effects for health or capable of improving physiological functions. By “food supplement”, is meant a foodstuff having the purpose of completing normal food diet. A food supplement is a concentrated source of nutrients or other substances having a nutritional or physiological effect, when they are taken alone or as a combination in small amounts. According to the invention, “functional food” summarizes foodstuff and corresponding products lately developed to which importance is attributed not only due to them being valuable as to nutrition and taste but due to particular ingredient substances. In some embodiments, the composition typically comprises carriers or vehicles. “Carriers” or “vehicles” mean materials suitable for administration and include any such material known in the art such as, for example, any liquid, gel, solvent, liquid diluent, solubilizer, or the like, which is non-toxic and which does not interact with any components of the composition in a deleterious manner. Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. In some embodiments, the composition comprises any other ingredients or excipients known to be employed in the type of composition in question. Non limiting examples of such ingredients include: proteins, amino acids, carbohydrates, oligosaccharides, lipids, prebiotics or probiotics, nucleotides, nucleosides, other vitamins, minerals and other micronutrients.

As used herein, the term “therapeutically effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease.

In some embodiments, the BCFA of the present invention are administered to the patient in combination with another active ingredient. In some embodiments, the BCFA are administered to the patient in combination with at least one nutrient selected from the group consisting of glutamine, arginine, tryptophan, leucine, isoleucine, valine, omega-3 PUFA, vitamin D, and curcumin. In some embodiments, the BCFA are administered in combination with an anti-TNFα drug. As used herein, the term “anti-TNFα drug” is intended to encompass agents including proteins, antibodies, antibody fragments, fusion proteins (e.g., Ig fusion proteins or Fc fusion proteins), multivalent binding proteins (e.g., DVD Ig), small molecule TNFα antagonists and similar naturally- or non-naturally-occurring molecules, and/or recombinant and/or engineered forms thereof, that, directly or indirectly, inhibit TNFα activity, such as by inhibiting interaction of TNFα with a cell surface receptor for TNFα, inhibiting TNFα protein production, inhibiting TNFα gene expression, inhibiting TNFα secretion from cells, inhibiting TNFα receptor signalling or any other means resulting in decreased TNFα activity in a subject. The term “anti-TNFα drug” preferably includes agents which interfere with TNFα activity. Examples of anti-TNFα drugs include, without limitation, infliximab (REMICADE™, Johnson and Johnson), human anti-TNF monoclonal antibody adalimumab (D2E7/HUMIRA™, Abbott Laboratories), etanercept (ENBREL™, Amgen), certolizumab pegol (CIMZIA®, UCB, Inc.), golimumab (SIMPONI®; CNTO 148), CDP 571 (Celltech), CDP 870 (Celltech), as well as other compounds which inhibit TNFα activity, such that when administered to a subject in which TNFα activity is detrimental, the disorder (i.e. acute severe colitis) could be treated.

The invention will be further illustrated by the following figures and examples. However, these examples and figures should not be interpreted in any way as limiting the scope of the present invention.

### FIGURES

FIG. 1: BCFAs trigger hyperSUMOylation of intestinal proteins in CACO2 cells Quantification of SUMO-conjugated proteins (above 50 kDa) in CACO2 cells exposed or not to BCFAs. Values are expressed as fold-change versus untreated cells (mean±s.e.m.; n=3-5; *, P<0.05; **, P<0.01 versus untreated cells; One-way ANOVA, with Dunnett's correction).

FIG. 2: BCFAs and SCFAs dampen responses to TNFα in intestinal cells. A, Quantification of IL8 and CCL20 mRNA levels in CACO2 cells pre-treated or not for 1 hour with BCFAs or SCFAs (either in their acidic or basic form) and then incubated for 1 hour with 100 ng/mL TNFα. Values are expressed as fold change versus untreated cells (mean s.d.; n=3-4; *, P<0.05 vs TNFα alone; **, P<0.01; ***, P<0.001; One-way ANOVA, with Dunnett's correction). B, Immunoblot analysis of IκBα and actin levels in CACO2 cells pre-incubated for 1 hour with 5 mM BCFAs or SCFAs, or incubated at a pH set to 5.2, and then stimulated for 30 min with 100 ng/mL TNFα. C, Quantification of IκBa levels, expressed as percentage of IκBa levels in untreated cells (mean±s.d.; n=3; *, P<0.05 vs TNFα alone; **, P<0.01; One-way ANOVA, with Dunnett's correction) (IB^{ic}, isobutyric acid; IB^{ate}, sodium isobutyrate; IV^{ic}, isovaleric acid; IV^{ate}, sodium isovalerate; Butic, butyric acid; Butate, sodium butyrate). D, TransEpithelial Electrical Resistance (TEER) in CACO2 cells grown to confluence and pre-treated or not for 1 hour with BCFAs and then incubated for 24 hours with 100 ng/mL TNFα. Values are expressed as TEER percent variations compared with cells before TNFα treatment (mean±s.e.m.; n=9; **, P<0.01; NS, not significant; Student's t-test).

FIG. 3. BCFAs dampen inflammation and intestinal hyperpermeability in a mouse model of colitis A, Quantification of fecal calprotectin in untreated mice (CTRL) or mice treated with 2% DSS±150 mM sodium isobutyrate (IB) in drinking water (mean±s.e.m.; n=4-7; *, P<0.01, **, P<0.001; One-way ANOVA, with Tukey's correction). B, Evaluation of colonic permeability (mean±s.e.m.; n=4-5; **, P<0.01, ***, P<0.001; One-way ANOVA, with Tukey's correction).

### EXAMPLE

### Material & Methods

### Animals

Animal care and experimentation were approved by a regional Animal Experimentation Ethics Committee (APAFIS #21102-2019061810387832 v2 and APAFIS #16184-2018071711054339) and complied with the guidelines of the European Commission for the handling of laboratory animals (Directive 2010/63/EU). All efforts were made to minimize suffering of animals.

Eight-weeks-old C57Bl/6JRj male mice (Janvier Labs, Le-Genest-Saint-Isle, France) were housed at 23° C. (5 animals/cage) with a 12-h light-dark cycle in regular open cages. All animals were fed with a non-sterilized standard rodent diet (3430.PM.S10, Serlab, France). Drinking water was not sterilized. Animals were acclimatized to the animal facility for 1 week before experimentations.

To monitor the effects of gut bacteria depletion, animals were split in two groups (5-10 animals/group): one group had no antibiotic treatment and were gavaged once a day with drinking water, while the other group received antibiotics by oral gavage once a day. For oral gavages, mice received a volume of 10 μL/g body weight of drinking water supplemented with 0.1 mg/mL Amphotericin-B, 10 mg/mL Ampicillin, 10 mg/mL Neomycin trisulfate salt hydrate, 10 mg/mL Metronidazole and 5 mg/mL Vancomycin hydrochloride (Tirelle et al., 2020). This solution was delivered with a stainless steel tube without prior sedation of the mice. To prevent fungal overgrowth in the antibiotic-treated animals, mice were pre-treated with Amphotericin-B for 3 days before the beginning of the protocol (Tirelle et al., 2020). As for antibiotic treatment, Amphotericin-B was delivered by oral gavage (10 μL/g body weight of drinking water supplemented with 0.1 mg/mL Amphotericin-B) (Tirelle et al., 2020).

To monitor the effect of BCFAs on chemically-induced intestinal inflammation, animals were split in three groups (8 animals/group): one group had no treatment, one group was treated with DSS (Dextran Sulfate Sodium, MB Biomedicals, France; 2% in drinking water) from D4 to D11 to induce colitis and one group was treated both with sodium isobutyrate (150 mM in drinking water, pH 7.4; from DO to D13) and DSS (D4 to D11).

At the end of each study, all animals were euthanized by an intraperitoneal injection of an overdose of ketamine (200 mg/kg body weight) and xylazine (20 mg/kg body weight). Cessation of heartbeats and non-responsiveness to noxious stimulus (hind paw pinch) were used as criteria to verify death. Segments from the jejunum, the caecum and the colon were then removed, as well as cecal contents, frozen in liquid nitrogen and stored at −80° C.

Protein Extraction from Mouse Intestinal Tissues

Intestinal tissues were mechanically lysed using bead beating in a buffer containing 50 mM HEPES pH 8.0, 8 M urea buffer, supplemented with 10 mM N-ethyl-maleimide (NEM; SUMO protease inhibitor). Tissue lysates were then centrifugated for 15 min at 13,000×g at 4° C. Supernatents were collected, mixed with one volume of Laemmli buffer (125 mm Tris-HCl [pH 6.8], 4% SDS, 20% glycerol, 100 mm dithiothreitol [DTT], 0.02% bromphenol blue) and analyzed by immunoblotting.

### Cell Culture

CACO2 (American Type Culture Collection (ATCC)-HTB-37), HeLa (ATCC-CCL2) and T84 (ATCC CCL-248) cells were cultivated at 37° C. in a 5% CO₂ atmosphere. CACO2 and HeLa cells were cultivated in Minimum Essential Medium (MEM) (Eurobio) supplemented with 2 mM L-Glutamine (Invitrogen), 10% Fetal Bovine Serum (FBS, Eurobio), non-essential aminoacids (Sigma), 1 mM sodium pyruvate (Gibco) and a mixture of penicillin (10000 U/mL) and streptomycin (10 mg/mL). T84 cells were cultivated in DMEM/F12 (Dulbecco's Modified Eagle Medium F-12) (Eurobio) supplemented with 10% FBS and 2.5 mM L-Glutamine. CACO2 and T84 cells were seeded in wells at a density of 1.1×10⁵ cells/cm² and 1.7×10⁵ cells/cm², respectively, the day before incubation with BCFAs or SCFAs.

Before treatments, cell culture medium was replaced by HBSS (Hanks' Balanced Salt Solution; Sigma). Cells were then treated as indicated in the text and directly lysed with Laemmli buffer. For BCFAs and SCFAs treatments, 100 mM stock solutions in water were first prepared from the corresponding acidic form (e.g. isobutyric acid) or from the sodium salt of the corresponding basic form (e.g. sodium isobutyrate) and then further diluted in cell culture media (HBSS). When needed, the pH of cell culture medium was set using either 0.1 M NaOH or 0.1 M HCl solution. For treatments with ROS inhibitors, CACO2 cells were pre-incubated for 30 min with 5 mM N-acetyl-cysteine (NAC) or 10 μM Diphenyleneiodonium (DPI) and then incubated for 1 h with 5 mM isobutyric acid or isovaleric acid. For TNFα treatments, CACO2 cells were first incubated with BCFAs or SCFAs for 1 hour and then incubated with 100 ng/mL recombinant human TNFα (PeproTech). For immunoblotting and qRT-PCR analyses, cells were lysed after 30 min or 1h of incubation with TNFα, respectively. For Transepithelial electrical resistance (TEER) measurements, cells were incubated for 24 h with TNFα. Viability of cells incubated with HBSS at various pH or with BCFAs was assessed using the CellTiter-Glo® luminescent cell viability assay (Promega), according to the manufacturer's protocol.

### Immunoblot Analyses

Cell lysates and protein extracts from intestinal tissues in Laemmli buffer were boiled for 5 min, sonicated and protein content was resolved using SDS-polyacrylamide gel electrophoresis. Proteins were then transferred on PVDF membranes (GE Healthcare) and detected after incubation with specific antibodies using ECL Clarity Western blotting Substrate (BioRad). All displayed immunoblots are representative of at least three independent experiments. Quantifications of proteins were performed on a ChemiDoc Imaging System (Bio-rad). SUMO2/3-conjugated proteins levels (above 50 kDa), SUMO1-conjugated protein levels (above 50 kDa), and other specific protein levels were normalized either by the level of total proteins above 50 kDa (determined using the TGX-stain free imaging technology; Bio-rad) or by the level of actin in each lysate.

### Detection of Reactive Oxygen Species

Detection of ROS was adapted from (Kim et al., 2019). Luminol was dissolved in NaOH 0.1 M to obtain a 50 mM stock solution. A stock solution of 1000 U/mL HRP (HorseRadish Peroxidase) was prepared in parallel in PBS (Phosphate-Buffered Saline). After treatment with BCFAs or SCFAs or incubation in HBSS at pH 5.2, CACO2 and HeLa culture media were collected and centrifugated for 5 min at 13,000×g at room temperature. The pH of these culture media was then buffered to 7.5 to avoid pH-dependent interferences with luminol activity. Luminol (1 mM final concentration) and HRP (4 U/mL) were finally added to each culture media and luminescence was quantified immediately on a luminometer (Tecan).

### Evaluation of DeSUMOylase Activity

DeSUMOylase activity assays were adapted from (Kunz et al., 2019). For in vitro cell lines, CACO2 and T84 cells grown in 12-well plates were scraped in 100 μL lysis buffer (Tris HCl pH 8.0 50 mM, EDTA 5 mM, NaCl 200 mM, Glycerol 10%, NP40 0.5%). For in vivo tissues, caecal segments were resuspended in lysis buffer (800 μL for 100 mg of tissues), mechanically lysed using bead beating and further diluted 25 times in lysis buffer. Negative controls were prepared by adding 10 mM N-ethymaleimide (NEM; Sigma-Aldrich) to cell lysates. Recombinant human SUMO1-AMC and SUMO2-AMC proteins (R&D Systems) were diluted in parallel to 500 nM in Assay buffer (Tris HCl pH 8.0 50 mM, Bovine Serum Albumin (BSA) 100 μg/mL, Dithiothreitol (DTT) 10 mM). For each measurement, 10 μL of cell or tissue lysates were mixed with 40 μL of SUMO-AMC containing Assay buffer and fluorescence (λ_{Ex}=380 nm; λ_{Em}=460 nm) was recorded for 30 min at 37° C. on a Flexstation 3 microplate reader (Molecular Devices). DeSUMOylase activities were determined by calculating the initial rate of fluorescence emission in each lysate and by normalizing by the quantity of proteins in the corresponding sample, determined in parallel using BCA assays (Pierce™ BCA Protein Assay Kit).

### Cell Fractionation

Fractionation of CACO2 cells incubated or not with 5 mM isobutyric, isovaleric or butyric acids for 5 h was performed with the Subcellular Protein Fractionation Kit (Thermo Scientific), according to the manufacturer's protocol. Extracts corresponding to cytosolic, nuclear soluble, and chromatin-bound fractions were collected and mixed 1:1 with Laemmli buffer. The remaining insoluble pellets, corresponding to the nuclear matrix, were resuspended directly in Laemmli buffer. All fractions were then boiled for 5 min and sonicated before immunoblotting analyses.

### Evaluation of Intracellular pH

CACO2 and T84 cells, grown in 96-well plates were loaded with 2 μM BCECF-AM (2′,7′-Bis-(2-Carboxyethyl)-5-(and-6)-Carboxyfluorescein, Acetoxymethyl Ester; Invitrogen) for 30 min in HBSS at 37° C. BCECF is a fluorescent dye exhibiting a pH-dependent excitation profile (Liang et al., 2007). Cells were then washed twice and incubated in HBSS with or without BCFAs or with HBSS at definite pH. Cell fluorescence (λ_{Emission}535 nm) was recorded at two different excitation wavelengths: 440 nm and 490 nm. Comparison of the ratios of fluorescence intensities (490/440 nm) was performed to detect changes in intracellular pH. Calibration solutions (with pH ranging from 4.5 to 7.5) were used as controls (Intracellular pH Calibration Buffer Kit; Molecular Probes).

### Quantification of Proinflammatory Cytokine Expression

Total RNAs were extracted from CACO2 cells using the RNeasy Plus Mini kit (Qiagen) following manufacturer's instructions. For each condition, 1 μg of total RNAs was reverse transcribed using random hexamers and M-MLV reverse transcriptase (Invitrogen). Specific cDNAs were then quantified by qPCR using Itaq Universal SYBR Green Supermix (BioRad) on a Mastercycler ep Realplex system (Eppendorf, Hamburg, Germany). GAPDH was used as an internal reference for normalization. Serial dilution of target cDNAs were included on each plate to generate a relative curve and to integrate primer efficiency in the calculations.

### Evaluation of Intestinal Epithelial Permeability

CACO2 cells were seeded in Transwell inserts and cultivated for 21 days. Monolayer formation and differentiation was monitored by daily evaluation of transepithelial electrical resistance (TEER) measurement, performed with an EVOM epithelial voltohm meter equipped with “chopstick” electrodes. After three weeks, cell culture media were replaced by HBSS. Cells were then preincubated or not with isobutyric or isovaleric acids for 1 hour. 100 ng/mL TNFα was then added to both apical and basolateral compartments. TEER was evaluated after 24h of incubation.

### Fecal Calprotectin Quantification

Fecal pellets were weighted and resuspended in 600 μL of PBS with 1% protease and phosphatase inhibitors (Sigma-Aldrich, USA). After a centrifugation step (12000×g, 15 min, 4° C.), calprotectin was quantified in the obtained supernatants using the S100A8 DuoSet® kit (R&D Systems, Minneapolis), according to the manufacturer's protocol.

### Intestinal Permeability

Colon samples were cut along the mesenteric border. Colonic permeability was assessed by measuring Lucifer yellow (440 Da; Sigma-aldrich) fluxes in Ussing chambers with an exchange surface of 0.07 cm2 (Harvard Apparatus, Holliston, MA). Lucifer yellow (250 μg/ml) was added to the mucosal side. After 3 h at 37° C., medium from the serosal side was removed and the fluorescence level of Lucifer yellow (excitation: 428 nm; emission: 540 nm) was quantified.

### Results

### Gut Microbiota Depletion Decreases SUMO2/3 Protein Conjugation in the Caecum.

In order to evaluate the potential impact of the gut microbiota on intestinal SUMOylation, we compared the global SUMOylation patterns of different intestinal segments either from conventional mice or from mice with a depleted gut microbiota. Depletion of mice intestinal bacteria was performed via the oral gavage of a cocktail of antibiotics during 7 days (Tirelle et al., 2020). The SUMOylation patterns of segments from the jejunum, the caecum and the colon were then analyzed by immunoblotting experiments using anti-SUMO1 and anti-SUMO2/3 antibodies (data not shown). The level of SUMO-conjugated proteins (above 50 kDa) was quantified in each sample (data not shown). Interestingly, we observed that mice with a depleted gut microbiota exhibit a significant decrease in the level of SUMO2/3-conjugated proteins in the caecum (data not shown). This decrease is specific to the SUMO2/3 isoform as the caecal level of SUMO1-conjugated proteins is not modified in response to antibiotics treatment. This decrease in SUMO2/3-conjugated protein levels is furthermore specific to the caecum as we did not observe any significant modification of the SUMOylation patterns in the jejunum or colon of mice treated with antibiotics (data not shown).Together, these results suggest that the gut microbiota regulates protein SUMOylation in the caecum.

### BCFAs Trigger HyperSUMOylation in Intestinal Cell In Vitro.

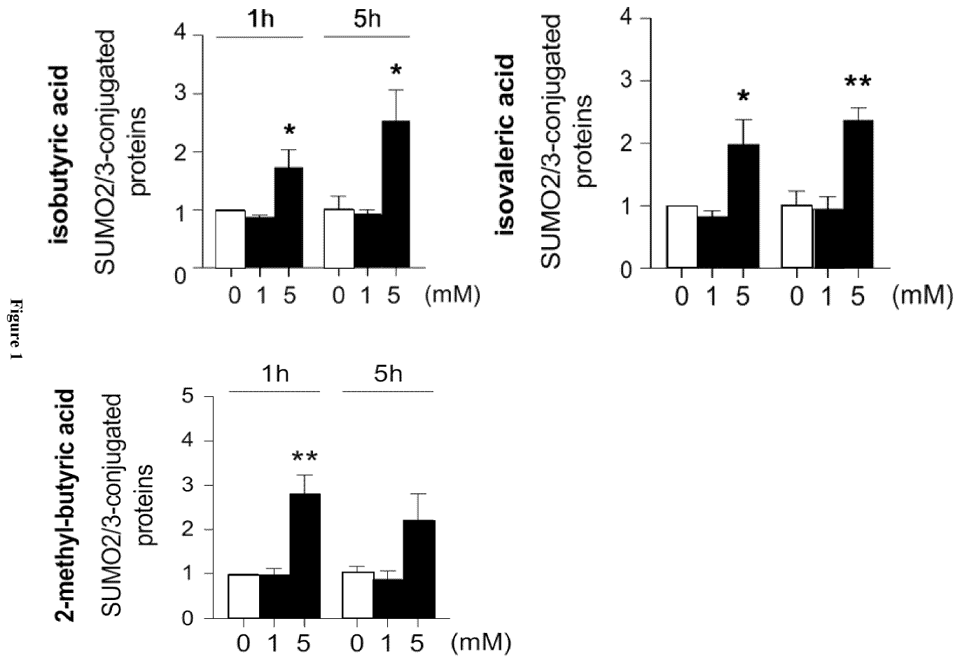

As fatty acids such as SCFAs and BCFAs are important mediators of the interactions between host and gut bacteria, we assessed whether these metabolites modulate host intestinal cell SUMOylation. We first monitored the effect of BCFAs on intestinal cell SUMOylation in vitro by incubating CACO2 or T84 cells cells for 1h or 5 h with isobutyric, isovaleric or 2-methyl-butyric acids (1 mM or 5 mM final concentrations) (FIG. 1). Interestingly, we observed that all BCFAs induced a significant increase in the level of proteins conjugated to SUMO2/3 after 1h or 5h of incubation (at 5 mM concentration) in CACO2 cells.

This hyperSUMOylation is similarly observed in T84 cells, after 1h of incubation with 5 mM isobutyric, isovaleric acids or 2-methyl-butyric acids (FIG. 1). In contrast to SUMO2/3-conjugated proteins, the pattern of proteins conjugated to SUMO1 is not affected by BCFAs in CACO2 cells (FIG. 1). Of note, the concentrations of BCFAs used here do not decrease cell viability (data not shown).

To determine whether the hyperSUMOylation induced by BCFAs is reversible, CACO2 cells were incubated with 5 mM isovaleric acid for 1h and then washed and allowed to recover in BCFA-free culture medium for 1 or 4h. We observed that the initial hyperSUMOylation triggered by isovaleric acid rapidly disappears after the removal of this BCFA (data not shown).

Together, these results demonstrate that BCFAs specifically increase SUMO2/3 conjugation in intestinal cell lines. This is consistent with our previous observations in vivo in which microbiota depletion leads to a specific decrease in SUMO2/3 conjugation in the caecum of mice (data not shown).

### The Effect of BCFAs on Intestinal SUMOylation is pH Dependent

BCFAs are weak organic acids, which exist in solution either as acidic (R—COOH) or basic (R—COO⁻) forms. For example, addition of 5 mM isobutyric acid in HBSS medium leads to a pH of ˜5.2 with approximatively 28% (i.e. ˜1.5 mM) of isobutyric acid and 72% (i.e. ˜3.5 mM) of isobutyrate. In contrast, addition of 5 mM sodium isobutyrate in HBSS medium leads to a solution with a pH of ˜7.5 containing approximatively 0.2% (i.e. ˜0.01 mM) of isobutyric acid and 99.8% (i.e. ˜4.99 mM) of isobutyrate. To decipher whether both acidic and basic forms of BCFAs can trigger hyperSUMOylation in intestinal cells, we added 5 mM isovaleric acid to CACO2 cell culture medium and increased gradually the cell culture medium pH from 5.2 to 7.0 (thereby decreasing the relative concentration of isovaleric acid and increasing those of isovalerate) (data not shown). We did not observe any significant hyperSUMOylation when cells where incubated in these conditions at 7.0, in contrast to cells incubated at pH 5.2. This suggests that only isovaleric acid (and not isovalerate) promotes SUMO-conjugation on intestinal proteins.

We then added increasing amounts of isovaleric acid to CACO2 cell culture media and set in parallel the pH of this medium between 5.2 and 7.0. For each pH, the amount of isovaleric acid added to cells was calculated to maintain the final concentration of isovaleric acid in the cell culture medium of ˜1.4 mM. We observed that this increase in BCFA concentration restores the hyperSUMOylation observed in CACO2 cells at pH6 and 7 (data not shown). This result demonstrates that BCFAs, when present in high concentration, trigger hyperSUMOylation even at neutral or weakly acidic pH.

To decipher if the hyperSUMOylation induced by isovaleric acid is only due to the associated acidification of the extracellular milieu, we compared the SUMOylation pattern of CACO2 cells incubated for 1, 2 or 5 h with 5 mM isovaleric acid (pH 5.2) or with culture medium without isovaleric acid and with a pH set to 5.2 (data not shown). We observed that the hyperSUMOylation triggered by isovaleric acid cannot be recapitulated by an equivalent acidic pH (data not shown).

Of note, the acidic forms of fatty acids are uncharged and freely diffusible across cellular membranes, in contrast to the basic forms, which are negatively charged and can only cross membranes thanks to specific transporters. Thus, as only the acidic forms of BCFA induce an hyperSUMOylation of intestinal proteins, we can hypothetise that these forms diffuse passively across the cell membrane, and then act intracellularly on intestinal cell SUMOylation.

To decipher whether the entry of the acidic forms of BCFAs inside cells may alter the intracellular pH (pHi) (as these acidic forms may release protons once inside cells), we monitored pHi in CACO2 and T84 cells using a pH-sensitive fluorescent dye (BCECF-AM) (Liang et al., 2008). Interestingly, we observed a significant decrease in pHi in both CACO2 and T84 cells incubated with 5 mM isobutyric and isovaleric acids (data not shown). As an acidic extracellular milieu may also decrease pHi, we compared the pHi measured in CACO2 cells incubated with various concentrations of isovaleric acid (at definite pH) with the one obtained for cells incubated in culture medium at similar pH (data not shown). In all conditions, the decrease in pHi triggered by isovaleric acid is greater than the one triggered by the corresponding acidic condition (data not shown). Similar results were obtained in T84 cells (data not shown). These results strongly suggest that the acidic forms of BCFAs diffuse inside intestinal cells where they trigger a decrease in the intracellular pH. This decrease is more efficient than the one induced by an acidic extracellular milieu, probably because the plasma membrane is more permeable to the acidic forms of BCFAS than to protons alone.

We finally monitor the level of SUMO2/3-conjugation in CACO2 cells incubated for 5h in culture medium with acidic pH. We did not observe any significant increase in SUMOylation for cells incubated in pH ranging from 7.5 down to 5.2, thereby confirming our previous observations (data not shown). Further decreasing the extracellular pH eventually leads to an increase in CACO2 cells SUMOylation, which is probably linked to a stronger decrease of CACO2 cells intracellular pH, as in the case of incubation with isovaleric acid (data not shown).

Of note, incubating CACO2 and T84 cells in culture medium with pH ranging from 7.5 to 5.0 does not decrease cell viability, nor the expression level of E1, E2 or E3 SUMO enzymes (data not shown).

### SCFAs Also Affect Intestinal SUMOylation.

To complete our results obtained with BCFAs, we determined whether SCFAs similarly impact intestinal cell SUMOylation. We incubated CACO2 cells with acetic, butyric and propionic acid for 5h (pH˜5.2; 5 mM final concentration). Interestingly, we observed that SCFAs also induce a significant increase in the level of SUMO2/3-conjugated proteins (data not shown). In contrast, incubation of cells with sodium acetate, butyrate or propionate (pH˜7.5; 5 mM final concentration) does not trigger any changes in the SUMOylation pattern of CACO2 cells. Together, these results indicate that SCFA, as observed with BCFA, modulate intestinal protein SUMOylation in a pH-dependent manner (data not shown).

BCFAs-Induced hyperSUMOylation is Dependent of ROS Production

Butyric acid was previously reported to induce ROS (Reactive Oxygen Species) production in both IEC-6 intestinal epithelial cells and HeLa cells (Kumar et al., 2009). We thus tested whether SCFAs and BCFAs similarly induce ROS production in CACO2 cells. For this, we used a sensitive luminol-based ROS detection assay (Kim et al., 2019). We observed that the addition of isobutyric, isovaleric or butyric acid induce ROS production in CACO2 cells after 1h of incubation (data not shown). This oxidative stress is transient as the level of ROS was less important after 5h of incubation (data not shown). Interestingly, the oxidative stress induced by BCFAs and SCFAs is pH-dependent as no ROS were detected after incubation with sodium isobutyrate, isovalerate or butyrate (data not shown). This suggests that ROS are produced only in response to the diffusion of the acidic form of BCFAs and SCFAs inside CACO2 cells and to the associated drop in intracellular pH. We confirmed this hypothesis by showing that ROS production induced by 5 mM isovaleric acids is greater than the one observed with cells incubated in the corresponding acidic conditions (data not shown).

To determine whether ROS production was responsible for the previously observed hyperSUMOylation triggered by fatty acids, we pre-incubated CACO2 cells with two ROS scavengers, N-acetyl cysteine (NAC) and Diphenyleneiodonium (DPI). These cells were then incubated with isobutyric or isovaleric acids for 1h. We observed that preincubation with these oxidative stress inhibitors significantly block BCFAs-induced hyperSUMOylation (data not shown). Together, these results demonstrate that the acidic forms of SCFAs and BCFAs trigger the production of ROS in intestinal cells, which in turn promotes SUMO-conjugation of intestinal proteins.

BCFAs Inhibit Intestinal Cells deSUMOylases

Global increase in SUMOylation could result either from an increase in the SUMOylation machinery's activity or from an inhibition of cellular deSUMOylases. As deSUMOylases were reported to be sensitive to oxidative stress (Stankovic-Valentin and Melchior, 2018; Xu et al., 2008), we evaluated whether BCFAs could inhibit SUMO-deconjugation in intestinal cells. For this, CACO2 and T84 cells were incubated with isobutyric or isovaleric acids and lysed. Cell lysates were then mixed with SUMO1 or SUMO2 peptides covalently linked to AMC (7-amino-4-methylcoumarin). The activity of deSUMOylases was then quantified in these cell lysates by measuring the fluorescence intensity of AMC released after the deSUMOylase-dependent cleavage of the amide bond between AMC and SUMO (data not shown). Samples in which N-ethylmaleimide (NEM) was added were used as negative controls (Kunz et al., 2019). We demonstrated that incubation with 5 mM isobutyric or isovaleric acids for 5 h significantly decrease the initial rate of SUMO deconjugation reactions in cell lysates, both for SUMO1- and SUMO2-AMC substrates (data not shown). These results indicate that deSUMOylases are inhibited in response to BCFAs exposure.

Of note, we quantified in parallel the expression levels of E1 and E2 SUMO enzymes in CACO2 cells treated with BCFAs using immunoblotting experiments. We observed that isobutyric and isovaleric acids do not alter the level of SAE1/SAE2 or UBC9 (data not shown). Together, these results suggest that the hyperSUMOylation induced by BCFAs result from the inhibition of intestinal cell deSUMOylases.

To complete these results, we quantified the activity of deSUMOylases in the caecum of mice treated with antibiotics. Interestingly, we observed a significant increase in the activity of deSUMOylases in mice with a depleted gut microbiota, which nicely correlates with the observed decrease in the level of SUMO-conjugated proteins in these intestinal segments (data not shown). Together, these results highlight that the activity of intestinal deSUMOylases can be regulated by gut microbiota-derived metabolites.

### BCFAs/SCFAs-Induced ROS do not Affect Cullin-1 Neddylation in CACO2 Cells

In addition to SUMOylation, other Ubiquitin-like proteins such as NEDD8 were reported to be sensitive to oxidative stress. Previous reports established that ROS produced in response to butyric acid exposure inactivate the NEDD8-conjugating enzyme Ubc12 and trigger the loss of cullin-1 neddylation in HeLa cells (Kumar et al., 2007; Kumar et al., 2009). We thus assessed whether BCFAs also decrease cullin-1 neddylation in CACO2 or HeLa cells. Interestingly, we observed that isobutyric and isovaleric acid triggers cullin-1 deneddylation after 5h of incubation in HeLa cells but not in CACO2 cells (data not shown). This suggests that the consequences of SCFAs/BCFAs-induced ROS are cell-type dependent and that these fatty acids do not affect neddylation in CACO2 cells.

### BCFAs and SCFAs Promote SUMOylation of Chromatin-Bound Proteins

In order to determine whether proteins conjugated to SUMO in response to SCFAs/BCFAs are located in specific cellular compartments, we performed cell fractionation assays. We isolated proteins from cytosolic, nuclear soluble and chromatin-associated fractions as well as proteins from the so-called nuclear matrix (a nuclear fraction characterized by its insolubility and resistance to high salt and nuclease extractions, in which several SUMO targets and enzymes, such as PML or PIASy, are accumulating; Sachdev et al., 2001, Ribet et al., 2017). SUMO2/3-conjugated proteins in basal conditions (without SCFAs/BCFAs) were mainly observed in nuclear fractions (nuclear soluble and chromatin fractions), as expected since many SUMO targets are known to be nuclear (Zhao et al., 2018). Very interestingly, we observed that the level of SUMO-conjugated proteins is strongly increased in the nuclear matrix fraction in response to SCFAs/BCFAs (and slightly decreased in the nuclear soluble fraction) (data not shown). These results highlight that SCFAs/BCFAs promotes the SUMOylation of nuclear factors, which are associated with the nuclear matrix.

BCFAs/SCFAs-Induced hyperSUMOylation Impair NF-κB Inflammatory Responses

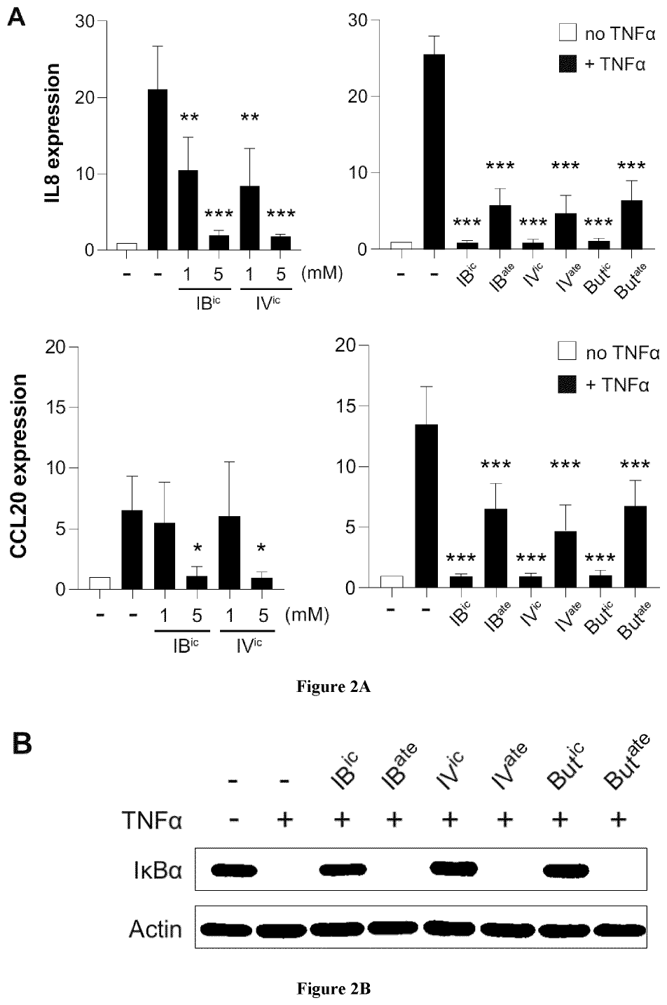

As SUMOylation is known to regulate inflammatory responses (Boulanger et al., 2021; Karhausen et al., 2021), we determined whether BCFAs/SCFAs-induced hyperSUMOylation modulate inflammatory responses in intestinal cells. To do so, we incubated CACO2 cells with TNFα in the presence or absence of BCFAs. We then quantified the expression levels of the pro-inflammatory IL8 and CCL20 cytokines by qRT-PCR. We observed that both isobutyric and isovaleric acids downregulate the transcription of IL8 and CCL20 in response to TNFα (FIG. 2A). We then compared the respective effect of the acidic or basic forms of BCFAs and SCFAs on the expression of these cytokines. We observed that the basic form of BCFAs and SCFAs partially decrease expression of IL8 and CCL20. Interestingly, we show that the acidic forms of BCFAs and SCFAs further decrease the expression of IL8 and CCL20 to the level observed in cells unstimulated by TNFα (FIG. 2A). As acidic forms are triggering hyperSUMOylation in contrast to basic forms of SCFAs/BCFAs, these results strongly suggest that hyperSUMOylation contributes to pro-inflammatory cytokines downregulation in intestinal cells, although SCFAs/BCFAs-mediated SUMO-independent mechanisms might also be involved.

As IL8 and CCL20 expression is regulated by the NF-κB transcription factor, we tested whether BCFAs could interfere with the NF-κB signaling pathway. To do so, we focused on the degradation of the IκBα inhibitor, which is a key step in the activation of NF-κB and a pre-requisite for NF-κB translocation into the nucleus. We quantified using immunoblotting experiments the level of IκBa in CACO2 cells incubated with TNFα in the presence or absence of BCFAs and SCFAs. We observed that isobutyric, isovaleric and butyric acids block the TNFα-triggered degradation of IκBa (FIGS. 2B and 2C). This inhibition was not observed with sodium isobutyrate, isovalerate and butyrate, again indicating that the hyperSUMOylation induced by the acidic forms of SCFAs/BCFAs dampen the NF-κB signaling pathway (FIGS. 2B and 2C).

### BCFAs Promote Intestinal Epithelial Integrity

We finally determined whether BCFAs regulate intestinal permeability. To do so, CACO2 cells were grown for 3 weeks in Transwell systems in order to reconstitute an in vitro model of differentiated intestinal epithelium. Cells were then incubated with TNFα and the permeability of the obtained epithelium was monitored by measuring the transepithelial electrical resistance (TEER) between the apical and basal compartments. Treatment of these epithelia with TNFα for 24h induce a significant decrease in TEER, which corresponds to an increase in epithelial permeability, as previously described (FIG. 2D). Interestingly, we show that co-incubation of CACO2 cells with isobutyric and isovaleric acids block this TNFα-induced increase in epithelial permeability. This result show that BCFA promote epithelial integrity in response to inflammatory stimuli.

### Oral Administration of BCFA Dampens Inflammation and Intestinal Hyperpermeability in a Mouse Model of Colitis

In order to evaluate the anti-inflammatory effects of BCFA in vivo, we performed a pilot study to assess the effect of oral administration of isobutyrate in a mouse model of colitis.

To do so, mice were treated with 2% DSS in drinking water for 7 days to induce colitis. Mice were treated or not in parallel with 150 mM sodium isobutyrate in drinking water.

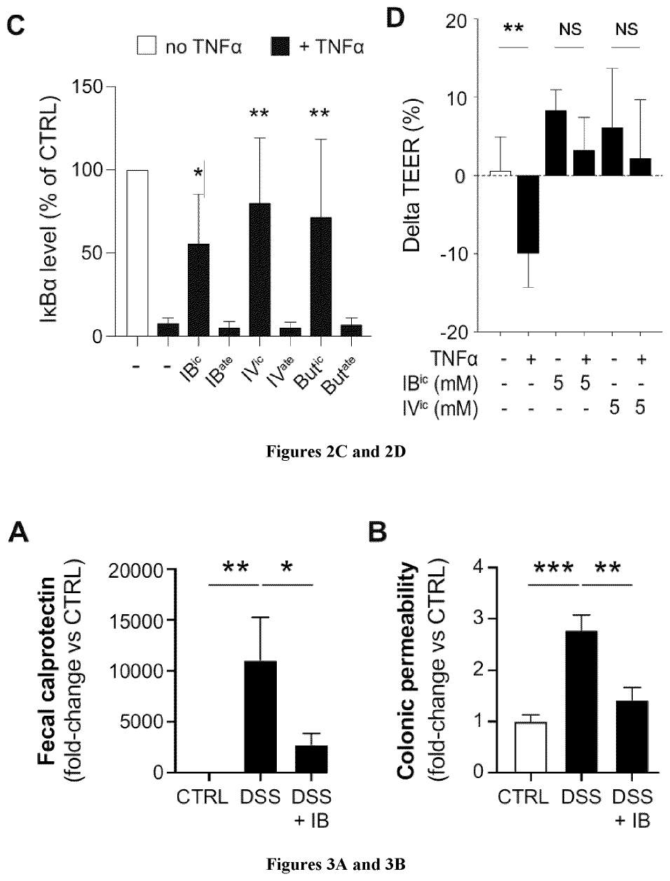

Intestinal inflammation was evaluated by quantifying calprotectin in fecal pellets 3 days after the end of the DSS treatment. Mice treated with DSS exhibit a strong inflammation characterized by increased levels of fecal calprotectin. Interestingly, addition of isobutyrate in drinking water significantly decreases the level of fecal calprotectin in DSS-treated mice (FIG. 3A).

We evaluated in parallel intestinal barrier integrity by measuring colonic permeability using Ussing chambers. Mice treated with DSS exhibit a significant increase in colonic permeability three days after the end of the DSS treatment. Interestingly, addition of isobutyrate in drinking water significantly decreases this DSS-induced colonic hyperpermeability (FIG. 3B). Together, these preliminary results suggest that oral administration of BCFA dampens intestinal inflammation triggered by DSS and decreases the associated intestinal hyperpermeability.

### Discussion:

Post-translational modifications are widely used by eukaryotic cells to modulate rapidly, locally and specifically the interactions or activities of key proteins. SUMOylation plays an essential role in intestinal physiology and more particularly in epithelial integrity maintenance, by controlling cell renewal and differentiation, as well as mechanic stability of the epithelium (Demarque et al., 2011; Karhausen et al., 2021). Not surprisingly, several pathogens were shown to manipulate intestinal SUMOylation in order to interfere with the activity of key host factors involved in infection (Ribet and Cossart, 2018). Most of these pathogens are decreasing SUMOylation, using independent mechanisms, which illustrates a nice example of evolutive convergence. In contrast to pathogens, the potential impact of gut commensal bacteria on SUMOylation has not been investigated. Here, we show that bacterial metabolites upregulate intestinal SUMOylation by controlling the activity of host deSUMOylases. As the SUMOylation level of a given target results from the dynamic equilibrium between conjugation and deconjugation reactions, the inactivation of deSUMOylases result in the increase in protein SUMOylation levels. Interestingly, we identified that the proteins SUMOylated in response to BCFAs/SCFAs are mainly associated with the nuclear matrix. As many SUMO targets are transcription factors, we can hypothesize that BCFAs/SFAs-induced SUMOylation modifies intestinal cell gene expression (Boulanger et al., 2021) Our result show that BCFAs/SCFAs dampen inflammatory responses of intestinal cells by upregulating SUMOylation. SCFAs, and more particularly butyrate, has already been shown to modulate intestinal inflammation (Parada Venegas et al., 2019). The potential effect of BCFAs on inflammation remain in contrast poorly documented. Interestingly, long-chain BCFAs (with more than 14 carbons) were shown to decrease the expression of IL8 in response to LPS in CACO2 cells and to decrease the incidence of necrotizing enterocolitis in a neonatal rat model (Yan et al., 2017; Ran-Ressler et al., 2011). Whether these effects are triggered by the acidic form of these long-chain BFCA, once translocated inside intestinal cells, remain to be determined. Of note, lactic acid, which is abundantly produced by the vaginal microbiota, also elicits anti-inflammatory responses on human cervicovaginal epithelial cells (Hearps et al., 2017). Interestingly, only lactic acid, and not lactate, prevents pro-inflammatory cytokines expression in epithelial cells, which nicely echoes our result on the anti-inflammatory properties of the acidic forms of BCFAs/SCFAs on intestinal cells (Hearps et al., 2017; Delgado-Diaz et al., 2019). The vaginal pH being naturally acid (pH<4), lactic acid is the predominant form in this environment. In the case of BCFAs and SCFAs produced by gut microbiota, the intraluminal pH is varying depending on the intestinal segment. This pH ranges from 5.5-7.5 in the caecum/right colon and then increases in the left colon and rectum to 6.1-7.5 (Nugent et al., 2001). Even though the acidic forms of SCFAs/BCFAs are not predominant in these conditions, the physiological high concentrations of SCFAs/BCFAs (i.e. ˜100 mM for SCFAs) may be high enough to have a concentration of protonated fatty acids sufficient to modulate intestinal SUMOylation.

SUMOylation has been involved in intestinal diseases such as Inflammatory Bowel Diseases (IBD). Indeed, patients with IBD show a downregulation of the UBC9 enzyme and a decrease in SUMOylated protein levels in the colon, which correlates with disease severity (Mustfa et al., 2017). These SUMO alterations, which can also be observed in a mouse model of colitis, were proposed to contribute to intestinal immune responses deregulation (Mustfa et al., 2017). This hypothesis is supported by the partial inhibition of gut inflammation observed in response to PIAS1 E3 ligase overexpression in the intestine and the associated increase in SUMOylation (Yavvari et al., 2019). Our results suggest that BCFAs/SCFAs may similarly limit inflammation in this context, by restoring SUMOylation in intestinal cells.

In conclusion, this work unveils a new mechanism used by the gut microbiota to modulate intestinal cell activities and dampen inflammation. It highlights in addition the therapeutic potential of SUMOylation in targeting inflammatory diseases such as IBD or IBS.

### REFERENCES

Throughout this application, various references describe the state of the art to which this invention pertains. The disclosures of these references are hereby incorporated by reference into the present disclosure.
Blaak, E. E., Canfora, E. E., Theis, S., Frost, G., Groen, A. K., Mithieux, G., Nauta, A., Scott, K., Stahl, B., van Harsselaar, J., et al. (2020). Short chain fatty acids in human gut and metabolic health. Benef Microbes 11, 411-455.
Blachier, F., Mariotti, F., Huneau, J. F., and Tome, D. (2007). Effects of amino acid-derived luminal metabolites on the colonic epithelium and physiopathological consequences. Amino Acids 33, 547-562.
Boulanger, M., Chakraborty, M., Tempe, D., Piechaczyk, M., and Bossis, G. (2021). SUMO and Transcriptional Regulation: The Lessons of Large-Scale Proteomic, Modifomic and Genomic Studies. Molecules 26.
Cappadocia, L., and Lima, C. D. (2018). Ubiquitin-like Protein Conjugation: Structures, Chemistry, and Mechanism. Chem Rev 118, 889-918.
Chang, H. M., and Yeh, E. T. H. (2020). SUMO: From Bench to Bedside. Physiol Rev 100, 1599-1619.
Delgado-Diaz, D. J., Tyssen, D., Hayward, J. A., Gugasyan, R., Hearps, A. C., and Tachedjian, G. (2019). Distinct Immune Responses Elicited From Cervicovaginal Epithelial Cells by Lactic Acid and Short Chain Fatty Acids Associated With Optimal and Non-optimal Vaginal Microbiota. Front Cell Infect Microbiol 9, 446.
Demarque, M. D., Nacerddine, K., Neyret-Kahn, H., Andrieux, A., Danenberg, E., Jouvion, G., Bomme, P., Hamard, G., Romagnolo, B., Terris, B., et al. (2011). Sumoylation by Ubc9 regulates the stem cell compartment and structure and function of the intestinal epithelium in mice. Gastroenterology 140, 286-296.
Flotho, A., and Melchior, F. (2013). Sumoylation: a regulatory protein modification in health and disease. Annu Rev Biochem 82, 357-385.
Gasaly, N., de Vos, P., and Hermoso, M. A. (2021). Impact of Bacterial Metabolites on Gut Barrier Function and Host Immunity: A Focus on Bacterial Metabolism and Its Relevance for Intestinal Inflammation. Front Immunol 12, 658354.
Hearps, A. C., Tyssen, D., Srbinovski, D., Bayigga, L., Diaz, D. J. D., Aldunate, M., Cone, R. A., Gugasyan, R., Anderson, D. J., and Tachedjian, G. (2017). Vaginal lactic acid elicits an anti-inflammatory response from human cervicovaginal epithelial cells and inhibits production of pro-inflammatory mediators associated with HIV acquisition. Mucosal Immunol 10, 1480-1490.
Impens, F., Radoshevich, L., Cossart, P., and Ribet, D. (2014). Mapping of SUMO sites and analysis of SUMOylation changes induced by external stimuli. Proc Natl Acad Sci USA 111, 12432-12437.
Jaskiewicz, J., Zhao, Y., Hawes, J. W., Shimomura, Y., Crabb, D. W., and Harris, R. A. (1996). Catabolism of isobutyrate by colonocytes. Arch Biochem Biophys 327, 265-270.
Karhausen, J., Ulloa, L., and Yang, W. (2021). SUMOylation Connects Cell Stress Responses and Inflammatory Control: Lessons From the Gut as a Model Organ. Front Immunol 12, 646633.
Kim, J. S., Jeong, K., Murphy, J. M., Rodriguez, Y. A. R., and Lim, S. S. (2019). A Quantitative Method to Measure Low Levels of ROS in Nonphagocytic Cells by Using a Chemiluminescent Imaging System. Oxid Med Cell Longev 2019, 1754593.
Kimura, I., Ichimura, A., Ohue-Kitano, R., and Igarashi, M. (2020). Free Fatty Acid Receptors in Health and Disease. Physiol Rev 100, 171-210.
Koh, A., De Vadder, F., Kovatcheva-Datchary, P., and Backhed, F. (2016). From Dietary Fiber to Host Physiology: Short-Chain Fatty Acids as Key Bacterial Metabolites. Cell 165, 1332-1345.
Kumar, A., Wu, H., Collier-Hyams, L. S., Hansen, J. M., Li, T., Yamoah, K., Pan, Z. Q., Jones, D. P., and Neish, A. S. (2007). Commensal bacteria modulate cullin-dependent signaling via generation of reactive oxygen species. EMBO J 26, 4457-4466.
Kumar, A., Wu, H., Collier-Hyams, L. S., Kwon, Y. M., Hanson, J. M., and Neish, A. S. (2009). The bacterial fermentation product butyrate influences epithelial signaling via reactive oxygen species-mediated changes in cullin-1 neddylation. J Immunol 182, 538-546.
Kunz, K., Muller, S., and Mendler, L. (2019). Assays of SUMO protease/isopeptidase activity and function in mammalian cells and tissues. Methods Enzymol 618, 389-410.
Kunz, K., Piller, T., and Muller, S. (2018). SUMO-specific proteases and isopeptidases of the SENP family at a glance. J Cell Sci 131.
Lapaquette, P., Fritah, S., Lhocine, N., Andrieux, A., Nigro, G., Mounier, J., Sansonetti, P., and Dejean, A. (2017). Shigella entry unveils a calcium/calpain-dependent mechanism for inhibiting sumoylation. Elife 6.
Liang E, Liu P, Dinh S. Use of a pH-sensitive fluorescent probe for measuring intracellular pH of Caco-2 cells. Int J Pharm 2007; 338:104-9.
Mustfa, S. A., Singh, M., Suhail, A., Mohapatra, G., Verma, S., Chakravorty, D., Rana, S., Rampal, R., Dhar, A., Saha, S., et al. (2017). SUMOylation pathway alteration coupled with downregulation of SUMO E2 enzyme at mucosal epithelium modulates inflammation in inflammatory bowel disease. Open Biol 7.
Nugent, S. G., Kumar, D., Rampton, D. S., and Evans, D. F. (2001). Intestinal luminal pH in inflammatory bowel disease: possible determinants and implications for therapy with aminosalicylates and other drugs. Gut 48, 571-577.
Parada Venegas, D., De la Fuente, M. K., Landskron, G., Gonzalez, M. J., Quera, R., Dijkstra, G., Harmsen, H. J. M., Faber, K. N., and Hermoso, M. A. (2019). Short Chain Fatty Acids (SCFAs)-Mediated Gut Epithelial and Immune Regulation and Its Relevance for Inflammatory Bowel Diseases. Front Immunol 10, 277.
Ran-Ressler, R. R., Khailova, L., Arganbright, K. M., Adkins-Rieck, C. K., Jouni, Z. E., Koren, O., Ley, R. E., Brenna, J. T., and Dvorak, B. (2011). Branched chain fatty acids reduce the incidence of necrotizing enterocolitis and alter gastrointestinal microbial ecology in a neonatal rat model. PLoS One 6, e29032.
Ribet D, Lallemand-Breitenbach V, Ferhi O, Nahori M A, Varet H, de The H, et al. Promyelocytic Leukemia Protein (PML) Controls Listeria monocytogenes Infection. mBio 2017; 8.
Ribet, D., and Cossart, P. (2018). Ubiquitin, SUMO, and NEDD8: Key Targets of Bacterial Pathogens. Trends Cell Biol 28, 926-940.
Ribet, D., Hamon, M., Gouin, E., Nahori, M. A., Impens, F., Neyret-Kahn, H., Gevaert, K., Vandekerckhove, J., Dejean, A., and Cossart, P. (2010). Listeria monocytogenes impairs SUMOylation for efficient infection. Nature 464, 1192-1195.
Roediger, W. E. (1980). Role of anaerobic bacteria in the metabolic welfare of the colonic mucosa in man. Gut 21, 793-798.
Sivaprakasam S, Bhutia Y D, Yang S, Ganapathy V. Short-Chain Fatty Acid Transporters: Role in Colonic Homeostasis. Compr Physiol 2017; 8:299-314.
Sachdev S, Bruhn L, Sieber H, Pichler A, Melchior F, Grosschedl R. PIASy, a nuclear matrix-associated SUMO E3 ligase, represses LEF1 activity by sequestration into nuclear bodies. Genes Dev 2001; 15:3088-103.
Stankovic-Valentin, N., and Melchior, F. (2018). Control of SUMO and Ubiquitin by ROS: Signaling and disease implications. Mol Aspects Med 63, 3-17.
Tirelle, P., Breton, J., Riou, G., Dechelotte, P., Coeffier, M., and Ribet, D. (2020). Comparison of different modes of antibiotic delivery on gut microbiota depletion efficiency and body composition in mouse. BMC Microbiol 20, 340.
Verma, S., Mohapatra, G., Ahmad, S. M., Rana, S., Jain, S., Khalsa, J. K., and Srikanth, C. V. (2015). Salmonella Engages Host MicroRNAs To Modulate SUMOylation: a New Arsenal for Intracellular Survival. Mol Cell Biol 35, 2932-2946.
Xu, Z., Lam, L. S., Lam, L. H., Chau, S. F., Ng, T. B., and Au, S. W. (2008). Molecular basis of the redox regulation of SUMO proteases: a protective mechanism of intermolecular disulfide linkage against irreversible sulfhydryl oxidation. FASEB J 22, 127-137.
Yan, Y., Wang, Z., Greenwald, J., Kothapalli, K. S., Park, H. G., Liu, R., Mendralla, E., Lawrence, P., Wang, X., and Brenna, J. T. (2017). BCFA suppresses LPS induced IL-8 mRNA expression in human intestinal epithelial cells. Prostaglandins Leukot Essent Fatty Acids 116, 27-31.
Yavvari, P. S., Verma, P., Mustfa, S. A., Pal, S., Kumar, S., Awasthi, A. K., Ahuja, V., Srikanth, C. V., Srivastava, A., and Bajaj, A. (2019). A nanogel based oral gene delivery system targeting SUMOylation machinery to combat gut inflammation. Nanoscale 11, 4970-4986.
Zhao, X. (2018). SUMO-Mediated Regulation of Nuclear Functions and Signaling Processes. Mol Cell 71, 409-418.

1. A method of treating intestinal inflammation in a patient in need thereof comprising administering to the patient a therapeutically effective amount of branched chain fatty acids (BCFA).

2. The method of claim 1 wherein the patient suffers from intestinal inflammation associated with diseases selected from the group consisting of inflammatory bowel diseases such as ulcerative colitis, Crohn's disease, especially Crohn's disease in a state that affect specifically the colon with or without ileitis, microscopic colitis (lymphocytic colitis and collagenous colitis), infectious colitis caused by bacteria or by virus, radiation colitis, ischemic colitis, pediatric colitis, undetermined colitis, functional bowel disorders (and by extension functional digestive disorders) and other states of digestive microinflammation, including irritable bowel syndrome (described symptoms without evident anatomical abnormalities).

3. The method of claim 1 wherein the BCFA is a branched form of a fatty acid with a short acyl chain that comprises 3 to 7 carbons in the acyl chain.

4. The method of claim 3 wherein the BCFA is a branched form of a fatty acid selected from saturated or unsaturated fatty acids comprising 7 or less carbon atoms, 6 or less carbon atoms, 5 or less carbon atoms, 4 or less carbon atoms.

5. The method of claim 3 wherein the BCFA is selected from the group consisting of isobutyric, isovaleric, 2-methyl-butyric acids and mixtures thereof.

6. The method of claim 1 wherein the BCFA is used in its acidic form.

7. The method of claim 1 that comprises locally administering the BCFA to the rectum, colon and/or terminal ileum of the patient.

8. The method of claim 7 wherein the BCFA are administered orally, by means of a unit dosage form that selectively releases BCFA in the terminal ileum and/or colon of the patient.

9. The method of claim 7 wherein the BCFA are effectively administered to the colon by rectal administration of an enema formulation or rectal foam comprising the amount of BCFA.

10. The method of claim 7 wherein the BCFA are delivered to the ileum or colon of the patient by administration of an enterically coated unit dosage form.

11. The method of claim 1 wherein the BCFA are administered in the form of pharmaceutical composition.

12. The method of claim 1 wherein the BCFA are administered in the form of a nutritional composition.

13. The method of claim 1 wherein the BCFA are administered to the patient in combination with at least one nutrient selected from the group consisting of glutamine, arginine, tryptophan, leucine, isoleucine, valine, omega-3 PUFA, vitamin D, and curcumin.

14. The method of claim 1 wherein the BCFA are administered in combination with an anti-TNFα drug.
